(19) United States
(12) Patent Application Publication
(10) Pub. No.: US 2010/0069818 A1
(43) Pub. Date: Mar. 18, 2010

(54) Self-Stripping Hemodialysis Catheter

(76) Inventor: Harry R. Smouse, Peoria, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/559,728

(22) Filed: Sep. 15, 2009

Related U.S. Application Data
(60) Provisional application No. 61/096,892, filed on Sep. 15, 2008.

(51) Int. Cl. A61M 25/00; A61M 1/14

(52) U.S. Cl. 604/6.16

(57) ABSTRACT

A hemodialysis catheter is provided with an exterior surface that has two or more lumens, wherein at least one large hole extends through the exterior surface for dialysis and at least one small hole extends through the exterior surface, in proximity to the large hole. A first lumen serves as a blood outflow path from the arteriovenous system and a second lumen serves as an inflow path for blood to be returned to the system. The large holes provide for blood exchange and the small holes are strategically placed in proximity to and around the large holes and may comprise an angled wall so as to allow for a jetting of blood or a sterile solution at high force over the surface of the catheter that strips off the biofilm as it is formed on the exterior surface of the catheter.

### FIELD

The present invention relates generally to catheters. More particularly, the present invention relates to a hemodialysis catheter.

### BACKGROUND

Each year in the United States, thousands of patients experience kidney failure and require hemodialysis. Hundreds of thousands more already are in kidney failure and require hemodialysis on a permanent basis. Kidney failure may be broadly divided into two categories: acute renal failure and chronic renal failure. Acute renal failure is a rapidly progressive loss of kidney function, and may be reversible. Chronic renal failure can develop slowly, exist as the long term result of irreversible acute failure, or comprise part of a disease progression. Acute renal failure can be present on top of chronic renal failure. Some patients, such as those in acute renal failure, require dialysis for only a limited time, whereas others, in chronic renal failure, require life-long hemodialysis. Dialysis treatments replace some of the functions of the kidneys through waste removal and fluid removal. Blood flows by one side of a permeable membrane, and a dialysate or fluid flows by the opposite side. Smaller solutes and fluid pass through the membrane. The concentrations of undesired solutes (e.g. potassium, calcium, and urea) are high in the blood, but low or absent in the dialysis solution. Constant replacement of the dialysate ensures that the concentration of undesired solutes is kept low on one side of the membrane. One type of dialysis is hemodialysis. In hemodialysis, the patient's blood is pumped through the blood compartment of a dialyzer, exposing it to a semipermeable membrane. The cleansed blood is then returned through the circuit back to the body.

Traditionally, a method of dialysis used to treat patients in chronic renal failure is to connect an artery to a vein, either by using a surgically created fistula in the arm, or a synthetic graft placed under the skin. Both methods require needle punctures with the removal and return of blood back into the circulation.

A hemodialysis catheter is used when either of those accesses fails, or when the accesses are newly placed and have not healed enough to allow use. When either of those accesses fails, the hemodialysis catheter is used for exchanging blood to and from the hemodialysis machine from the patient. A hemodialysis catheter has advantages. Particularly, the patient does not have to endure a needle puncture to gain access to his or her arteriovenous system in every dialysis procedure; rather, the catheter can be periodically hooked up to the dialyzer system to provide a blood flow path between the patient and the dialyzer system. The catheter is placed into a large central vein, allowing for the rapid removal and return of blood back into the circulation. One hemodialysis catheter is used per patient and has two lumens; one lumen for the removal of blood and a second lumen for returning blood.

There are several common hemodialysis catheter malfunctions. First, fibrin sheath build-up occurs on the outside of the catheter. A fibrin sheath is an otherwise harmless biofilm that develops over time on catheters, impeding the flow of blood through the catheter. Additionally, the access vein may clot off once when the catheter is removed.

### SUMMARY

In accordance with the present invention, a catheter having a generally circular cross-section comprising an exterior surface, a first lumen, a second lumen, an upper end, a lower end, and a plurality of large holes for dialysis, wherein the plurality of large holes extends through the exterior surface into each of the first lumen and the second lumen, and a plurality of small holes, wherein the plurality of small holes are in proximity to the plurality of large holes, have a smaller radius than the plurality of large holes, and extend through the exterior surface into each of the first lumen and the second lumen. One lumen serves as a blood outflow path from the arteriovenous system and the other lumen serves as an inflow path for blood to be returned to the system. The small holes may be raised above and at an angle from the exterior surface of the catheter.

This catheter retards the development of a fibrin sheath on the exterior surface of the catheter by jetting a solution, in this case, blood, over the outside of the catheter, effectively stripping off the fibrin sheath. By retarding the development of a fibrin sheath the life of the catheter can be prolonged. Less manipulations of the catheter are required, allowing for less chance for error and procedural risk of injury to the patient. Additionally, less overall cost to the patient and hospital are required because the life of the catheter is prolonged.

### BRIEF DESCRIPTION OF THE DRAWINGS

Various embodiments are described herein with reference to the following drawings. Certain aspects of the drawings are depicted in a simplified way for reasons of clarity. Not all alternatives and options are shown in the drawings and, therefore, the invention is not limited in scope to the content of the drawings. In the drawings:

FIG. 1 is a perspective view of an exemplary embodiment of a two lumen hemodialysis catheter;

FIG. 2 is an enlarged section taken generally along line 1-1 in FIG. 1;

FIG. 3 is a perspective view of a portion of an exemplary embodiment of a catheter of the invention with two small holes for every large hole; and

FIG. 4 is a perspective view of an exemplary embodiment of a hemodialysis catheter of the invention with a third port.

### DETAILED DESCRIPTION

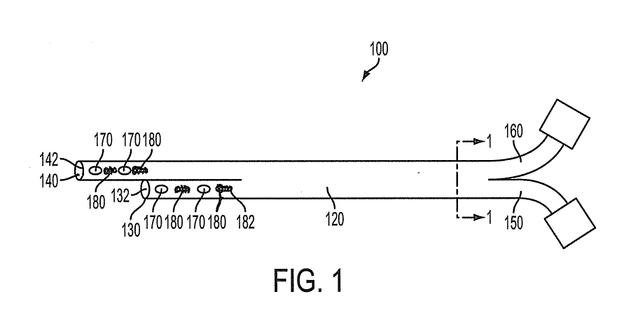

FIG. 1 depicts a perspective view of a catheter 100 according to an exemplary embodiment of the present invention. Catheter 100 is provided for use as a self-stripping hemodialysis catheter.

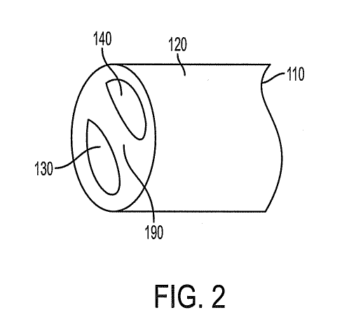

Catheter 100 includes an external circular cross section 110 (shown in FIG. 2), an exterior surface 120, a first lumen 130, a second lumen 140, an upper port 150, a lower port 160, a plurality of large holes 170, and a plurality of small holes 180. First lumen 130 is separated from second lumen 140 by a wall 190, as shown in FIG. 2. The invention is not limited to two lumens and any arrangement of multiple lumens may be provided. First lumen 130 terminates in a first end 132, while second lumen 140 terminates in a second end 142, as shown in FIG. 1. Upper port 150 may be for the removal of blood. Lower port 160 may be for the return of treated blood back into circulation.

Catheter 100 is shown in its unstressed configuration. Although catheter 100 is flexible, it can be straightened out for insertion and packaging. Catheter 100 is sized and shaped to fit into a human blood vessel so that the catheter resides in the blood vessel.

FIG. 2 shows a section of catheter 100 taken generally along line 1-1 of FIG. 1. First lumen 130 may comprise a generally elliptical cross-section. Alternatively, first lumen 130 may comprise a half-moon shaped cross-section. Second lumen 140 may comprise a generally elliptical cross-section. Alternatively, second lumen 140 may comprise a half-moon shaped cross-section. First and second lumens 130, 140 are not confined to these shapes and other cross-sections may be contemplated.

The large holes 170 are located toward the first and second ends 132, 142 of catheter 100 in FIG. 1. Although FIG. 1 shows two large holes 170 for each port, the invention is not limited to this number of large holes, and catheter 100 may comprise more or less large holes. Each large hole 170 extends through exterior surface 120 of catheter 100 into the lumen. A portion of the blood flowing through the lumen exits through each large hole 170. Typical rates of dialysis fluid flow are within the range of 300 to 500 milliliters per minute. Catheter 100 may be coated to retard infection.

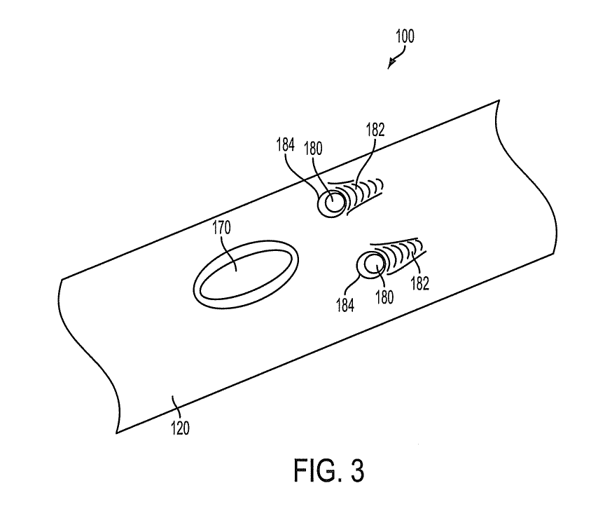

Catheter 100 also shows small holes 180 located near the large holes 170. Although FIG. 1 shows four small holes 180, the invention is not limited to this number, and catheter 100 may comprise more or less small holes. FIG. 3 shows a portion of catheter 100 with one large hole 170 and two small holes 180. As shown in FIG. 3, edge 182 along the perimeter on the exterior surface of each small hole 180 may be raised above the exterior surface 120. In the preferred apparatus, each small hole 180 is set at an angle to create angled wall 184 as shown in FIG. 3, which allows for blood or another solution to flow at a higher velocity over the exterior surface 120 of catheter 100. Edge 182 may also be set at an angle from exterior surface 120.

Blood is jetted at a high velocity over the exterior surface 120 in proximity to large holes 170. By jetting blood at high velocity over the exterior surface 120, any fibrin sheath build-up is stripped off the exterior surface and blood flow may continue to take place at the desired rate through large holes 170. The jetted blood or solution also retards the formation of fibrin on exterior surface 120.

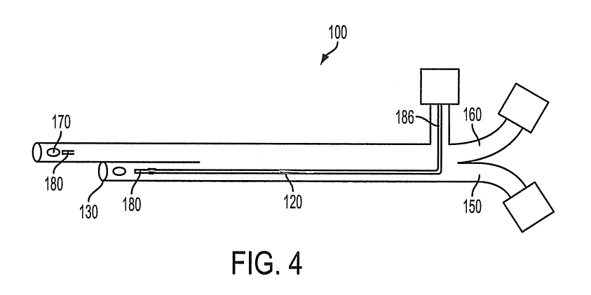

Small holes 180 may be positioned around each large hole 170, as shown in FIG. 1. Small holes 180 may extend through the return lumen of the dialysis catheter so that some of the returned blood is jetted over the catheter during each dialysis session. Alternative patterns and configurations for small holes 180 may be contemplated. One port may comprise a channel that extend through the length of the catheter, exiting through the small holes 180, so that fluid may flow through the channel and out the small holes. As another alternative, small holes 180 may extend through a third lumen 186 with a third port 188, as shown in FIG. 4. Prior to beginning dialysis, the dialysis machine may jet saline or another solution at velocity through third lumen 186 that is higher than the velocity of the flow of solution through large holes 170 over the catheter, stripping off the fibrin sheath, before beginning the actual dialysis treatment cycle. Blood may also flow through third lumen 186 and out small hole 180.

In operation, catheter 100 may be inserted into a patient's blood vessel, under local anesthesia. The catheter may be placed into the blood vessel by inserting a needle into the blood vessel, and placing a wire guide through the needle into the blood vessel. The needle is then removed from the wire guide and a dilator with an overlying sheath is slid over the wire guide into the blood vessel.

The hemodialysis process begins, and blood is removed from the patient through first lumen 130, through upper port 150, and into the dialysis machine. The processed blood is then sent from the dialysis machine to lower port 160, and through second lumen 140, where the blood is then re-introduced into the patient's blood vessel through large holes 170. Blood may also be re-introduced into the patient's blood vessel through the second end 142 of the catheter. Some of the blood that flows through each lumen will exit through each of the small holes 180. The blood that exits through small holes 180 will follow the path of angled wall 184 and will increase in velocity, flowing more rapidly as the flow hits exterior surface 120. In flowing across the exterior surface more rapidly, the flow will impact fibrin sheath that has built up on the exterior surface and the force of the flow will push the fibrin sheath off the exterior surface 120. The constant force of the flow across exterior surface 120 will also prevent fibrin sheath from building up on the surface.

Due to the high velocity jetting of blood over the exterior surface 120 that prevents build-up of a fibrin sheath, the life of catheter 100 will be prolonged. Additionally, catheter 100 will have improved function because of unimpeded large holes from fibrin sheath build-up, allowing for improved and shorter dialysis times. Prolonging the life of a catheter has many advantages: it results in fewer hospitalizations because a patient doesn't have to go in to the hospital and get the catheter switched as often, less risk of patient injuries due to fewer procedures, less expense, and fewer access site thromboses, or clotting off of the blood vessel due to the catheter presence.

This catheter may be used in a situation where a patient is newly diagnosed with renal failure and requires prolonged hemodialysis. In this situation, catheter 100 would be placed in the patient to allow for the treatment of the patient's blood. The catheter may be used when a dialysis patient has an existing dialysis catheter in place but the dialysis treatment cycles are slowing down. In this instance, the old catheter would be removed over a guidewire and catheter 100 of the present invention would be used to replace the failing catheter. In another situation, a dialysis patient may have an arteriovenous fistula or other permanent arm access in place that has failed and is not salvageable. A new arm access is planned and the patient will need catheter dialysis for several weeks or longer to bridge the patient until his or her arm access has matured. Catheter 100 may be placed in the patient in this situation.

It will thus be seen that certain changes may be made in the above constructions without departing from the spirit and scope of the invention. It is intended that all matter contained in the above description or shown in the accompanying drawings shall be interpreted as illustrative and not in a limiting sense.

We claim:

1. A catheter having a generally circular cross-section and an upper comprising: an exterior surface;
a first lumen;
a second lumen;
a first end;
a second end;
a plurality of large holes for dialysis, wherein each of the plurality of large holes extend through the exterior surface into each of the first lumen and the second lumen; and
a plurality of small holes, wherein each of the plurality of small holes is within proximity to each of the plurality of large holes, has a smaller radius than the each of the plurality of large holes, and extends through the exterior surface into each of the first lumen or the second lumen.

2. The catheter of claim 1, wherein the plurality of large holes and the plurality of small holes are located toward the first end and the second end of the catheter.

3. The catheter of claim 1, further comprising a third lumen.

4. The catheter of claim 3, wherein the third lumen is inside of the first lumen and wherein the third lumen exits into a small hole of the plurality of small holes.

5. The catheter of claim 1, wherein each of the plurality of small holes further comprises a perimeter edge that is raised above the exterior surface.

6. The catheter of claim 1, wherein each of the plurality of small holes comprises a wall surface, wherein the wall surface extends from the exterior surface of the catheter into either the first lumen or the second lumen.

7. The catheter of claim 6, wherein each of the plurality of small holes extends through the exterior surface to either the first lumen or the second lumen at an angle so that the wall surface is at an angle.

8. The catheter of claim 7, wherein at least a portion of the wall surface is beveled at an angle.

9. A self-stripping hemodialysis catheter comprising:
an exterior surface;
a first end;
a second end;
a plurality of lumens;
a plurality of first holes, wherein each of the plurality of first holes extends through the exterior surface into a lumen of the plurality of lumens;
a plurality of second holes, wherein the plurality of second holes comprises a smaller radius than the plurality of first holes, and extends through the exterior surface into a lumen of the plurality of lumens.

10. The hemodialysis catheter of claim 9, wherein the plurality of first holes and the plurality of second holes are located toward the first and second ends of the catheter.

11. The hemodialysis catheter of claim 9, wherein the plurality of lumens comprises two lumens.

12. The hemodialysis catheter of claim 11, wherein a second hole of the plurality of second holes further comprises a perimeter edge that is raised above the exterior 20 surface.

13. The hemodialysis catheter of claim 14, wherein each second hole of the plurality of second holes comprises a wall surface, wherein the wall surface extends from the exterior surface of the catheter to a lumen of the plurality of lumens.

14. The hemodialysis catheter of claim 15, wherein each second hole of the plurality of second holes extends through the exterior surface to a lumen of the plurality of lumens at an angle so that the wall surface is at an angle.

15. The hemodialysis catheter of claim 9, wherein the plurality of first holes extend perpendicularly through the exterior surface to a lumen of the plurality of lumens.

16. The hemodialysis catheter of claim 14, wherein at least a portion of the wall surface is beveled at an angle.

17. The hemodialysis catheter of claim 9, wherein the plurality of lumens comprises three lumens.

18. The hemodialysis catheter of claim 17, wherein one of the three lumens is inside another lumen of the three lumens and exits through a second hole of the plurality of second holes.
